(19) United States
(12) Patent Application Publication
(10) Pub. No.: US 2014/0010727 A1
(43) Pub. Date: Jan. 9, 2014

(54) Service and Maintenance Station for Handheld Drug Delivery Device

(71) Applicants: Michael Jugl, Frankfurt am Main (DE); Axel Teucher, Frankfurt am Main (DE)

(72) Inventors: Michael Jugl, Frankfurt am Main (DE); Axel Teucher, Frankfurt am Main (DE)

(73) Assignee: SANOFI-AVENTIS DEUTSCHLAND GMBH, Frankfurt am Main (DE)

(21) Appl. No.: 14/005,870

(22) PCT Filed: Mar. 22, 2012
(86) PCT No.: PCT/EP2012/055057

§ 371 Date: Sep. 18, 2013

(30) Foreign Application Priority Data
Mar. 22, 2011 (EP) 11159103.8

(51) Int. Cl. A61M 5/00

(52) U.S. Cl. CPC A61M 5/001
USPC 422/292; 134/166 R; 134/95.2; 134/105; 134/113; 134/115 R; 134/58 R

(57) ABSTRACT

The present invention relates to a service and maintenance station for a handheld drug delivery device of pen-injector type, comprising:

a receptacle section to receive at least a portion of the drug delivery device,

at least one service module adapted to maintain and/or to restore the operability of the drug delivery device, wherein the at least one service module comprises a cleaning and/or sterilizing module adapted to receive, to clean and/or to sterilize a distal end section of the drug delivery device to be connected with a piercing assembly.

### CROSS REFERENCE TO RELATED APPLICATIONS

The present application is a U.S. National Phase Application pursuant to 35 U.S.C. §371 of International Application No. PCT/EP2012/055057 filed Mar. 22, 2012, which claims priority to European Patent Application No. 11159103.8 filed Mar. 22, 2011. The entire disclosure contents of these applications are herewith incorporated by reference into the present application.

### FIELD OF INVENTION

The present invention relates to a service and maintenance station for a handheld drug delivery device, such as a pen-type injector.

It is therefore an object of the present invention to facilitate general handling, in particular the maintenance of drug delivery devices. It is a further aim to enhance patient safety when using the drug delivery device.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, in particular pen-type injectors have to meet a number of user-specific requirements. For instance, with patient's suffering chronic diseases, such like diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Moreover, a dose setting as well as a dose dispensing procedure must be easy to operate and has to be unambiguous.

Typically, such devices comprise a housing adapted to receive a cartridge at least partially filled with the medicament to be dispensed. The device further comprises a drive mechanism, typically having a displaceable piston rod which is adapted to operably engage with a piston of the cartridge. By means of the drive mechanism and its piston rod, the piston of the cartridge can be displaced in a distal direction and may therefore expel a pre-defined amount of the medicament via a piercing assembly to be releasably coupled with a distal end section of the housing of the drug delivery device.

Hence, the distal end section of such a drug delivery device, also denoted as cartridge holder, is adapted to hold and to receive the cartridge filled with the medicament. The cartridge holder typically comprises a threaded socket adapted to receive a double-tipped needle assembly. On the one hand, the needle assembly is adapted to penetrate or to pierce an elastic seal of the cartridge. On the other hand, the needle assembly, with its opposite tipped end, is intended to penetrate biological tissue in order to deposit the medicament in or beneath the skin of a patient.

Depending on the type of medicament, the cartridge and/or the entire drug delivery device have to be stored in a refrigerated environment. Furthermore, especially the distal end section of the drug delivery device and the interface section between needle assembly and cartridge holder have to be protected against dust, moisture and other environmental influences. Moreover, the cartridge holder-needle assembly interface should be clean or even sterilized.

Especially for elderly persons or for such patients suffering from impaired vision, visual control of the filling level of the cartridge via an inspection window provided in the cartridge holder section of the drug delivery device may become difficult.

It is therefore an object of the present invention to facilitate general handling, in particular the maintenance of drug delivery devices. It is a further aim to enhance patient safety when using the drug delivery device.

### SUMMARY

The present invention provides a service and maintenance station for a handheld drug delivery device, in particular for a pen-type injector. The service and maintenance station comprises a receptacle section adapted to receive at least a portion of the drug delivery device, e.g. of the pen-type injector. Preferably, the receptacle section is adapted to receive and to hold a distal portion of the housing of the drug delivery device, in particular a cartridge holder section.

The service and maintenance station further comprises at least one service module which is adapted to maintain and/or to restore the operability of the drug delivery device. The service and maintenance station and in particular its at least one service module serves to provide a semi- or fully automatic maintenance procedure or a respective check-up for the drug delivery device. Preferably, the maintenance station serves as a storage device for the drug delivery device. After use of the drug delivery device, it is to be placed in the receptacle section of the service and maintenance station until it is used again.

The at least one service module of the service and maintenance station further comprises a cleaning and/or sterilizing module. Said module is adapted to receive, to clean and/or to sterilize a distal end section of the drug delivery device, which is typically to be connected with a replaceable piercing assembly. The cleaning and/or sterilizing module may comprise a container at least partially filled with a cleaning and/or sterilizing agent.

The drug delivery device and the cleaning and/or sterilizing module may be displaceable with respect to each other in order to temporally plunge or to immerse the distal end section of the drug delivery device into the cleaning agent. The cleaning and/or sterilizing module may therefore provide an at least semi-automatic cleaning procedure for the distal end section of the drug delivery device.

In a preferred embodiment, the receptacle section comprises at least one fixing means for keeping the drug delivery device in a pre-defined service position. The fixing means provides a mechanical coupling and mechanical fixing of the drug delivery device in the receptacle section. The fixing means may also define a particular storage position or maintenance orientation of the drug delivery device with respect to the receptacle section of the service and maintenance station.

According to another embodiment, the service and maintenance station further comprises a fan and/or a heating module for drying and/or for sterilizing the distal end section of the drug delivery device. Preferably, cleaning and/or sterilizing of the distal end section of the drug delivery device is followed by a drying or heating procedure. Sterilization or cleaning as well as a subsequent drying or heating is preferably conducted right after the drug delivery device has been inserted into the receptacle section of the service and maintenance station. Additionally, the service and maintenance station may comprise a counter or a respective control unit adapted to sense and to monitor whether the drug delivery device is inserted into the service and maintenance station.

For instance, a cleaning and/or sterilizing procedure may not have to be conducted every time the drug delivery device is placed in the receptacle section. It may be sufficient, when e.g. every second or third insertion of the device into the receptacle section triggers a respective cleaning or sterilizing procedure. Additionally or alternatively, cleaning and/or sterilizing as well as a subsequent drying of the distal end section of the drug delivery device may be conducted on the basis of a pre-defined time schedule.

According to another preferred aspect, the at least one service module comprises a sensing arrangement adapted to determine a filling level of the cartridge placed in the drug delivery device and being filled with a medicament to be dispensed by said device. Preferably, the drug delivery device, in particular its cartridge holder comprises an inspection window, through which a filling level of the cartridge placed therein can be visually inspected. The service and maintenance station may comprise a sensing arrangement disposed along and/or around the inspection window. Said sensing arrangement typically comprises optical transmitters and respective detectors in order to determine the filling level of the cartridge in a contactless way.

For instance, a light source and a respective detector or detector array may be arranged in transmission or reflection geometry with respect to a transparent inspection window of the cartridge holder section of the drug delivery device.

In a further preferred embodiment, the at least one service module comprises a cooling member adapted to refrigerate the receptacle section of the service and maintenance station. This way, the service and maintenance station provides an effective cooling and refrigerating of the drug delivery device. The medicament contained therein can therefore be stored under appropriate thermal condition. The service and maintenance station may even provide a cooling and refrigerating function in a mobile environment. By way of the service and maintenance station providing refrigeration of the drug delivery device disposed therein, an effective cooling and refrigerating of the medicament can be provided at almost any time, in particular when the user is travelling. The cooling or refrigerating module might be electrically driven. It may comprise a Peltier element or a comparable electro-thermal converter.

The service and maintenance station may further be equipped with a charging module adapted to be electrically coupled with a re-chargeable battery of the drug delivery device. This way, a re-chargeable battery of the drug delivery device can be re-charged when the drug delivery device is disposed in the receptacle section of the service and maintenance station.

In a further preferred aspect, the service and maintenance station may be equipped with a battery module itself This way, the service and maintenance station becomes applicable for transportation and may provide appropriate thermal conditions for the drug delivery device even when not connected to an electricity network.

In still another preferred embodiment, the service and maintenance station further comprises an input/output interface for providing device-related information to a user and/or for receiving and for processing user commands. By way of the input/output interface, a wide range of user interaction scenarios can be implemented. The input/output interface may provide device-related information to a user, visually and/or acoustically. For instance, the input/output interface may indicate to a user, that the battery of the service and maintenance station has to be re-charged, that the filling level of the cartridge is too low, that cleaning and/or sterilizing of the drug delivery device is due and/or that the battery of the drug delivery device requires re-charging.

It is of further benefit, when the service and maintenance station comprises a control unit which is adapted to control a cleaning, a sterilizing, a re-charging and/or cooling of the drug delivery device when disposed in the receptacle section.

In general, the service and maintenance station may comprise only one service module providing at least some of the above described features. Alternatively, it is conceivable, that the service and maintenance station comprises a multiplicity of service modules, for instance a cleaning and/or sterilizing module, a drying module, a sensing module, a cooling module, a charging module as well as an input/output module. The control unit is typically adapted to communicate with at least some, preferably with all service modules provided in the service and maintenance station. The control unit may for instance trigger and control a cleaning and/or sterilizing procedure as well as a subsequent drying of the distal portion of the drug delivery device's housing.

In another aspect the service and maintenance station comprises a drug delivery device disposed in the receptacle section. Hence, the maintenance station is commercially distributed with a suitable drug delivery device. Service functionality of the maintenance station can therefore readily adapted to a particular type of drug delivery device to be exclusively used with said maintenance and service station.

It will be further apparent to those skilled in the pertinent art that various modifications and variations can be made to the present invention without departing from the spirit and scope of the invention. Further, it is to be noted, that any reference signs used in the appended claims are not to be construed as limiting the scope of the present invention.

### BRIEF DESCRIPTION OF THE DRAWINGS

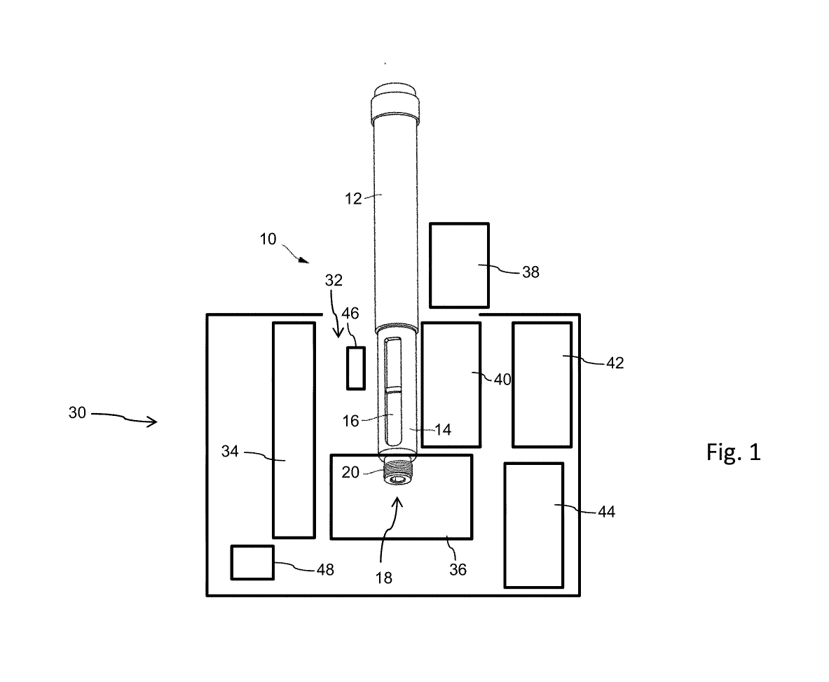

In the following description of the single FIGURE, a preferred embodiment of the invention will be described in detail, wherein FIG. 1 illustrates the service and maintenance station in a schematic and simplified way.

### DETAILED DESCRIPTION

The service and maintenance station 30 as indicated in FIG. 1 comprises a compact and box-like geometry. Towards its upper side, it comprises a receptacle 32 adapted to receive at least a distal portion 14 of a drug delivery device 10. The drug delivery device 10, which is illustrated as a pen-type injector comprises a proximal housing portion 12 and a distal housing portion 14, the latter being designed as a cartridge holder. The cartridge holder 14 further has an inspection window 16 in order to allow visual inspection of the filling level of a cartridge disposed therein. At its distal end section, the cartridge holder 14 comprises an interface 18 with a threaded socket 20. The threaded socket 20 is adapted to be coupled with a corresponding and disposable needle assembly, which is not explicitly illustrated here.

After usage of the drug delivery device 10, the needle assembly is typically unscrewed and discarded. With the detached needle assembly, the drug delivery device 10 is to be positioned in the receptacle 32. At the bottom portion of the receptacle 32, a cleaning and/or sterilizing module 36 is provided in order to clean and/or to sterilize the distal socket portion 18 of the drug delivery device 10. The cleaning and/or sterilizing module 36 may further comprise a drying and/or heating function which may be activated after a preceding cleaning or sterilizing procedure has been completed.

The service and maintenance station further comprises fixing means 46 in order to keep the drug delivery device 10 in a pre-defined position inside the receptacle 32. The fixing means 46 may comprise a releasable positive-fitting fixing means, such like a clip.

Additionally, the service and maintenance station may be equipped with a sensing unit or sensing module 34 aligned along the inspection window 16 of the cartridge holder 14. By way of the sensing arrangement 34, the filling level of the cartridge disposed in the cartridge holder can be detected in a contactless way, typically, by means of an optical transmitter and detection arrangement. Preferably, the detector provides a spatial resolution along the inspection window 16 in order to detect the filling level of the cartridge when the drug delivery device is appropriately disposed in the receptacle 32.

Additionally, the service and maintenance station may comprise a cooling or refrigerating module 40, which is adapted to actively refrigerate the receptacle 32 and thus the cartridge holder 14 of the drug delivery device 10. This way, particular thermal conditions required for storage of the medicament contained in the drug delivery device 10 can be provided. Furthermore, a risk of early deterioration of the medicament due to improper storage conditions can be minimized and patient safety can be enhanced.

The service and maintenance station 30 further has a charging module 38, which is adapted to re-charge a battery of the drug delivery device 10, which is for instance required to provide the drive mechanism of the device 10 with electrical power. Moreover, not only the drive mechanism but also some kind of display means of the drug delivery device 10 may require electric energy. By means of the charging module 38, an on-site battery of the drug delivery device 10 can be re-charged on a regular basis.

Not only the drug delivery device 10 but also the service and maintenance station 30 itself may be equipped with a re-chargeable battery 32. By means of a battery 42, the service and maintenance station 30 becomes particularly suitable for mobile applications. It may facilitate travelling and maintain required and appropriate storage conditions for the medicament even when connected to an electricity network.

By way of an input/output means 44, any device specific information gathered by any of the modules 34, 36, 38, 40, 42 can be indicated to a user, either visually and/or acoustically. For instance, the input/output module 44 may indicate the charging level of the battery 42. It may further illustrate the temperature of the cooling module 40 and/or of the receptacle 32. Additionally, it may display the filling level of the cartridge disposed in the cartridge holder 14 of the drug delivery device 10, which is detected by the sensing arrangement 34.

Moreover, the service and maintenance station is provided with a control unit 48 adapted to process any electrical signals generated by any one of the illustrated modules 34, 36, 38, 40, 42, 44. By way of the control unit 48, inter-module communication can be controlled.

Depending on the type of medicament, e.g. insulin or heparin, various parameters of the service and maintenance station 30, e.g. frequency of cleaning—and sterilizing procedures, storage temperatures and minimum filling level of the cartridge can be individually adapted according to pre-defined device—and/or medicament properties.

1-10. (canceled)

11. Service and maintenance station for a handheld drug delivery device of pen-injector type, comprising:
a receptacle section to receive at least a portion of the drug delivery device,
at least one service module adapted to maintain and/or to restore the operability of the drug delivery device, wherein the at least one service module comprises a cleaning and/or sterilizing module adapted to receive, to clean and/or to sterilize a distal end section of the drug delivery device to be connected with a piercing assembly.

12. The service and maintenance station according to claim 1, wherein the receptacle section comprises at least one fixing means for keeping the drug delivery device in a service position.

13. The service and maintenance station according to claim 11, further comprising a fan and/or a heating module for drying and/or sterilizing the distal end section of the drug delivery device.

14. The service and maintenance station according to claim 11, wherein the at least one service module comprises a sensing arrangement adapted to determine a filling level of a cartridge placed in the drug delivery device and being filled with a medicament to be dispensed by the drug delivery device.

15. The service and maintenance station according to claim 11, wherein the at least one service module comprises a cooling member adapted to refrigerate the receptacle section.

16. The service and maintenance station according to claim 11, further comprising a charging module adapted to be electrically coupled with a re-chargeable battery of the drug delivery device.

17. The service and maintenance station according to claim 11, further comprising a battery module.

18. The service and maintenance station according to claim 11, further comprising an input/output interface for providing device-related information to a user and/or for receiving and processing user-commands.

19. The service and maintenance station according to claim 11, further comprising a control unit adapted to control a cleaning, a sterilizing, a re-charging and/or cooling of the drug delivery device when disposed in the receptacle section.

20. The service and maintenance station according to claim 11, further comprising a drug delivery device disposed in the receptacle section.
